(19) United States
(12) Patent Application Publication
(10) Pub. No.: US 2015/0119756 A1
(43) Pub. Date: Apr. 30, 2015

(54) URINE SAMPLING DEVICE

(71) Applicant: SCA Hygiene Products AB, SE-405 03 Goteborg (SE)

(72) Inventors: Ulrika Husmark, Molnlycke (SE); Ingrid Gustafson, Asa (SE)

(21) Appl. No.: 14/389,124

(22) PCT Filed: Mar. 30, 2012
(86) PCT No.: PCT/SE2012/050358

§ 371 Date: Sep. 29, 2014

(51) Int. Cl. A61B 10/00; C12Q 1/04; A61F 5/44

(52) U.S. Cl. CPC A61B 10/007; A61F 5/4401; C12Q 1/04
USPC 600/573

(57) ABSTRACT

A urine sampling device including: a housing including a urine sampling chamber, the housing of the urine sampling device being arranged such that the urine sampling device can be associated with a hygiene absorbent product, and the urine sampling device includes an inlet for said fluid, wherein said chamber includes a dry gel that can be swelled by urine, and wherein said urine sampling device possesses the ability to preserve a bacteria content from a urine sampled in the device, either by means of the properties of the dry gel in itself and/or by providing a preservative in the dry gel. A hygiene absorbent product and a urine examination kit.

### CROSS REFERENCE TO RELATED APPLICATIONS

The present application is a U.S. National Stage Application of International Application No. PCT/SE2012/050358, filed on Mar. 30, 2012. The entire contents of International Application No. PCT/SE2012/050359 are hereby incorporated herein by reference in their entirety.

### TECHNICAL FIELD

An embodiment of the disclosure relates to a urine sampling device. An embodiment of the disclosure relates to a hygiene absorbent product comprising the urine sampling device and a urine examination kit.

### BACKGROUND

A document in the technical area of an embodiment of the disclosure is SE 532682 C2, where a diaper is described for collection of urine samples for diagnosing, e.g., urine tract infections. The urine from the user is guided through a feeding channel to a urine container located on the outside of the diaper. The urine container is dismountable.

### SUMMARY

An embodiment of the disclosure relates to sampling of urine from patients for later bacteria cultivation and analysis thereof. In particular, an embodiment of the disclosure assists with sampling from individuals where urination cannot be performed on command, such as elderly dement people or small children.

At least one of the problems is solved by an embodiment of the disclosure by a urine sampling device comprising: a housing comprising a urine sampling chamber, the housing of the urine sampling device being arranged such that the urine sampling device can be associated with a hygiene absorbent product, and the urine sampling device comprises an inlet for said fluid, wherein said chamber comprises a dry gel that can be swelled by urine, and wherein said urine sampling device possesses the ability to preserve a bacteria content in the urine sampled in the urine sampling device, either by means of the properties of the dry gel in itself and/or by providing a preservative in the dry gel.

The effect of an embodiment of the disclosure is that the sampling can be performed without any surveillance of a nurse. If it is desired to conduct the sampling at night, the urine sampling device can be added to the absorbent product and applied to the individual who is to be tested. The preservation of the bacteria makes it possible to leave the product on all night and in the morning the sampling device can be removed and the analysis conducted. This solves the problem of not being able to communicate with the individual from whom urine is to be sampled in that they do not need to signal when they have urinated and the sampling device can be left all night without the result of the urine sample will give a misleading result.

In accordance with a further development of an embodiment of the disclosure, said gel is a physically cross linked hydrophilic gel.

Gels of this type have good liquid retaining properties and are suitable for the aimed purpose of urine sampling.

In accordance with a further development of an embodiment of the disclosure, the gel is chosen from a group consisting of polysaccharides and synthetic polymers, preferably hydrophilic polymers.

The advantage of polysaccharides is that they come from a renewable source, which is considered beneficial for the environment, not least by the consumers. The advantage of synthetic polymers is that they are homogeneous in composition from batch to batch.

According to the said embodiment of said urine sampling device, said hydrophilic polymer is polyethyleneoxide (PEO).

The PEO polymer is particularly advantageous as it provides an inherent preservative effect of its own.

In accordance with a further development of an embodiment of the disclosure, said preservative is a composition that has acidic and/or desiccant properties.

In accordance with a further development of an embodiment of the disclosure, the preservative is chosen from a group consisting of acids such as boric acid and salts from these such as sodium borate or potassium borate.

This group of compounds has a proven preservative effect on bacteria causing urinary tract infection.

In accordance with a further development of an embodiment of the disclosure said preservative is present in an amount such that the number of bacteria trapped in the chamber should not change during at least 12 hours. The preservative should prevent growth and still not kill the bacteria. The preservative is good and precise enough for the intended use if it secures that growth is less than one log unit and/or death is less than one log unit.

There is an advantage if the preservative allows the sample to be held for a time period that will allow sampling for example over night, in particular for persons that cannot perform a sampling on command.

In accordance with a further development of the present invention an embodiment of the disclosure an outer surface of the urine sampling device comprises an attachment means, for attaching the urine sampling device to the body side of an absorbent product.

It is particularly advantageous to have a urine sampling device that can be attached easily to a standard hygiene absorbent product, without modifying it.

In accordance with a further development of an embodiment of the disclosure, said attachment means is arranged such that it can detachably attach the urine sampling device to said absorbent product.

This is particularly advantageous if the sampling person is not the same as the person that performs the analysis of the sampled urine. Then the urine sampling device can be detached for analysis in another location.

According to the said embodiment of the urine sampling device said attachment means is an adhesive, or a hook and loop arrangement.

These attachment means are particularly convenient as they are generally well recognized as having a good function together with hygiene absorbent products in general.

In accordance with a further development of an embodiment of the disclosure said chamber comprises a selected amount of dry gel, in order to swell and hold a predetermined volume of urine in the chamber.

By sampling a predetermined volume of urine the found bacteria can directly be counted and consistent analysing results is provided.

According to the said embodiment of the urine sampling device said volume is comprised in the interval 100 μl to 5000 μl preferably 150 μl to 500 μ.

These volume intervals have been found to be appropriate when examining and analysing urine.

In accordance with a further development of an embodiment of the disclosure, it comprises a closing device for closing said inlet to seal the chamber when said urine sampling device has received a predetermined volume of urine.

The advantage of providing a closing device is that the sampled urine is prevented from exiting once sampled, at least in any significant amount. Another advantage is that the urine sample stays in place when the urine sampling device is handled after sampling.

In accordance with a further development of an embodiment of the disclosure, the urine sampling device is dismountable by means of fractural impressions made in the housing, such that a urine sample absorbed by said gel can be removed from said chamber, for further examination.

The advantage of this is that the urine sample can be easier to extract from the device.

In accordance with a further development of an embodiment of the disclosure, the housing of the urine sampling device comprises a ventilation opening for ventilating air out of the urine sampling device when it is filled with urine.

The ventilation opening provides for a better filling of the device, as exiting air need not exit by the same opening as the urine entering.

An embodiment of the disclosure also comprises a hygiene absorbent product that comprises an associated urine sampling device according to what is mentioned above. In an alternative embodiment the hygiene absorbent product comprises means for incorporating the urine sampling device.

In some sampling situations it is preferred to provide a complete absorbent product when sampling the urine. It is then an advantage if the sampling person or the person to be investigated for urinary tract infections need not handle the device himself. And further the position of the device in the hygiene absorbent product is predetermined and thus sampling can be more consistent, than if the device is positioned in a position of choice.

An embodiment of the disclosure further comprises a urine examination kit, comprising a urine sampling device according the above and a sample collection container arranged to house the urine sampling device, for transport of said urine sampling device for further examination.

The urine examination kit is of advantage if sampling is performed far from a laboratory making the analysis. The sample in the urine sampling device can then be sent to the analyse laboratory.

The urine examination kit can further comprise a glove for preventing contamination of the urine sampling device when removing it from the hygiene absorbent product.

By providing a glove the handling of the urine sampling device is further improved in terms of contamination, risks.

### BRIEF DESCRIPTION OF THE DRAWINGS

FIG. 1 discloses a urine sampling device according to an embodiment of the disclosure,

FIG. 2 discloses the urine sampling device of FIG. 1 from another angle,

FIG. 3 discloses a hygiene absorbent product according to an embodiment of the disclosure,

FIG. 4 discloses a kit for sampling urine according to an embodiment of the disclosure,

FIG. 4a discloses a kit for sampling urine according to an embodiment of the disclosure,

FIG. 5 discloses a closing device of the inlet of a urine sampling device of an embodiment of the disclosure,

FIG. 5a discloses an alternative closing device of the inlet of a urine sampling device of an embodiment of the disclosure,

FIG. 6 discloses a urine sampling device of an embodiment of the disclosure having fractural impressions,

FIG. 7 discloses a hygiene absorbent product comprising the urine sampling device of an embodiment of the disclosure,

FIG. 8 Chart for example 1,

FIG. 9 First Chart for example 2,

FIG. 10 Second Chart for example 2.

### DETAILED DESCRIPTION

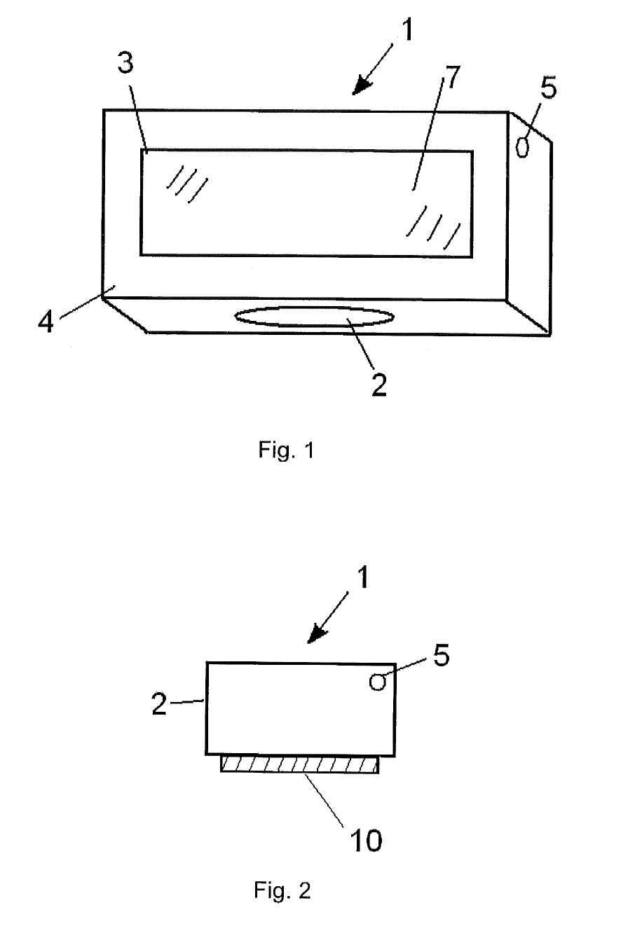

In FIG. 1 a first embodiment of a urine sampling device 1 according to an embodiment of the disclosure is disclosed. The urine sampling device 1 has a housing 4 comprising a chamber 3. The housing 4 is made in any suitable material. The housing 3 is preferably formed by a sheet of plastic or other suitable material such as paperboard made hydrophobic. The shape of the embodiment of FIG. 1 is a box. The shape of the urine sampling device 1 need not be a box. It can also have a cylinder shape and any other suitable three dimensional shapes. It can also be formed as a flat urine sampling device comprising the dry gel, wherein the urine sampling device can expand together with the gel. The chamber 3 can be of the size ranging from 100-5000 μl. The volume can be altered if needed for analytical reasons, for example 100 μl is thinkable as well as 200 μl or 400 μl. If the volume is too small the necessary amount of bacteria for a good analysis will not be present. A too large volume will make the device too large such that it will cause discomfort to the user during sampling.

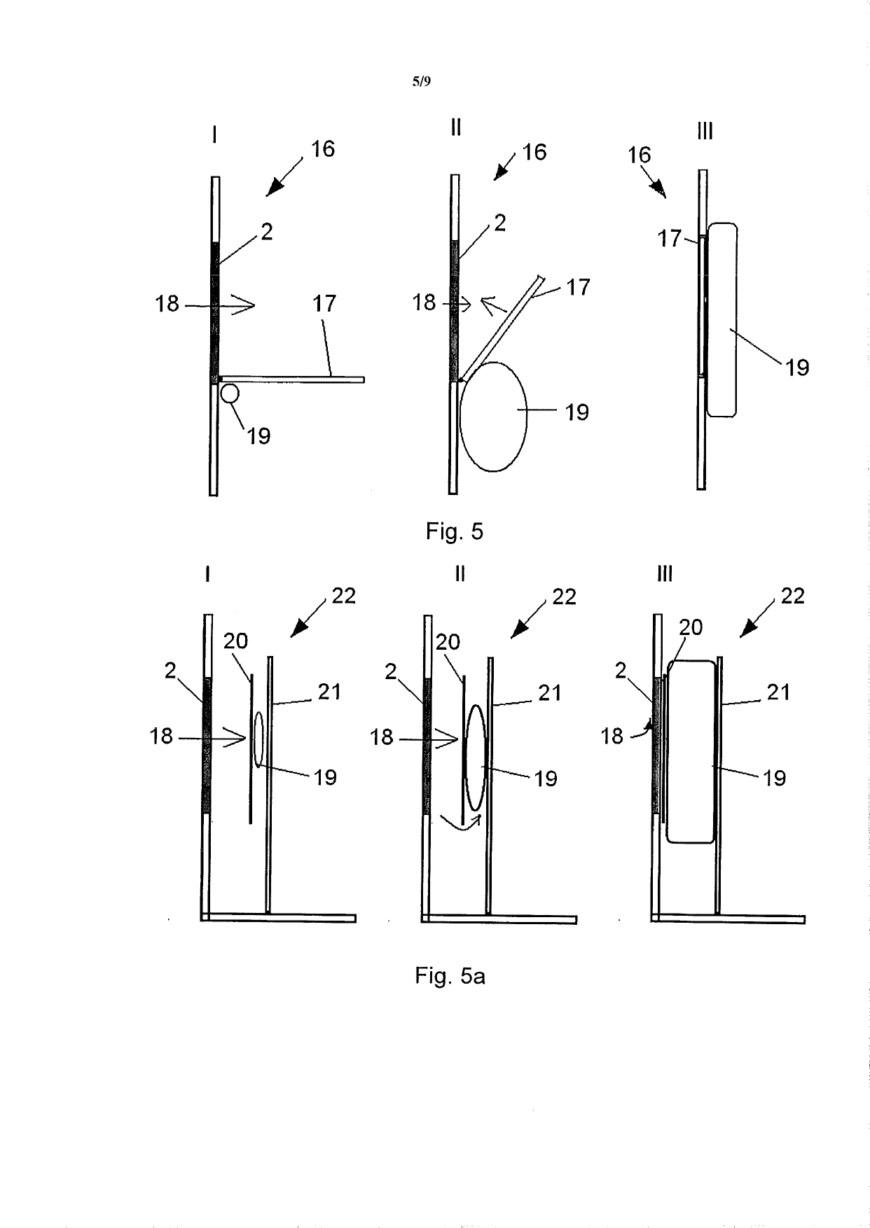

The inlet 2 of the urine sampling device 1 is provided with a feature that seals the chamber 3 after receiving the amount of urine that fills the chamber 3. The inlet 2 can be closed in the same way as described in the document WO 2008/131 904, i.e., where a swellable material closes the inlet 2. An alternative embodiment of the inlet closing can be seen in FIG. 5. The figure discloses a portion of the housing of the urine sampling device 1 comprising the inlet 2 wherein the inlet 2 is closable by means of a closing device 16 comprising a movable hatch 17. Urine enters in a filling sequence I-III, wherein the hatch 17 moves from a fully open position Ito a semi open position II to a closed position III. The movement of the hatch 17 is provided by a swelling material 19. The swelling material 19 can be any suitable material known to the person skilled in the art. The material 19 should have an ability to swell that does not interfere with the dry gel provided in the housing 4 for sampling. In FIG. 5a an alternative closing device 22 is disclosed where a slot formed by an inner wall 21 is provided. On the side of the wall 21 facing the inlet 2, a film 20 is provided. Sandwiched between the film 20 and the wall 22 is a swellable material 19 positioned. As urine enters, see arrow 18, it will partly penetrate behind the film 20 and the swellable material 19 will swell an thereby the film 20 will approach and close the inlet 2, in the sequence I-III. Swelling occurs in sufficiently slow manner such that the inlet 2 is not closed before the urine sampling device 1 is filled with urine.

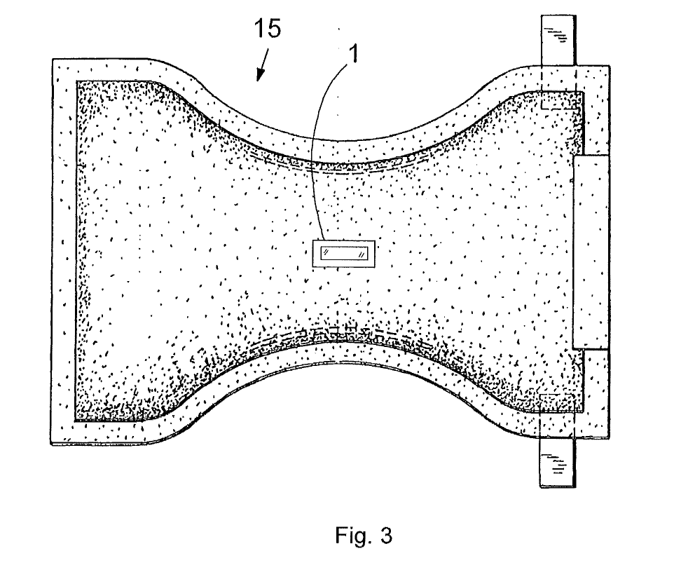

The housing 4 has preferably attachment means 10 added to one of its outer walls. The attachment means 10 can be any suitable attachment means such as an adhesive, or a hook and loop device. The attachment means 10 should be able to attach the urine sampling device 1 to a sheet of an absorbent hygiene product 15. The outer wall in question need not be fully provided with the attachment means 10, a portion of the housing 4 can provided with the attachment means 10. In a preferred embodiment the attachment means 10 is suitable for attaching it to a top sheet, i.e. the body side, of an absorbent product 15 as seen in FIG. 3. Even though the drawing discloses the urine sampling device 1 as attached in a position which is external to the top sheet, the urine sampling device 1 could also be fitted under said top sheet. Another position that is possible is inside the absorbent layer of the hygiene product 15. Such a positioning is preferred when a higher comfort to the user is preferred.

It is also possible to combine the urine sampling device 1 with an absorbent product 15 having a pocket 14 for residing the urine sampling device 1. See FIG. 7.

The urine sampling device 1 also can also be provided with a ventilation opening 5, for allowing air residing in the urine sampling device 1 to escape when the urine sampling device 1 receives the urine. The ventilation opening 5 need not be provided with a closing device 16 due to its size. However it could be provided with a closing device 16 as described above, or any other means for closing.

The urine sampling device 1 can also comprise a clear window 7. A urine sampling device 1 fitted with such a window 7 would be examinable before the gel is taken out, for example to see how well filled the urine sampling device 1 is, such that a new sample can be taken if the urine sampling device 1 is not properly filled. To further increase the ability of detecting if the urine sampling device is properly filled or not, an additive can be added to the urine sampling chamber which additive changes colour as urine enters the chamber. The colour change can be due to a colorant which is activateable by water solutions revealing a visual colour. An example of a suitable colorant is iron sulphate. A second possibility as to how a colour switch can be achieved is that if an acid is used as a preservative, such as boric acid, in combination with a pH indicator. Examples of suitable pH indicators are methyl red and cresol red.

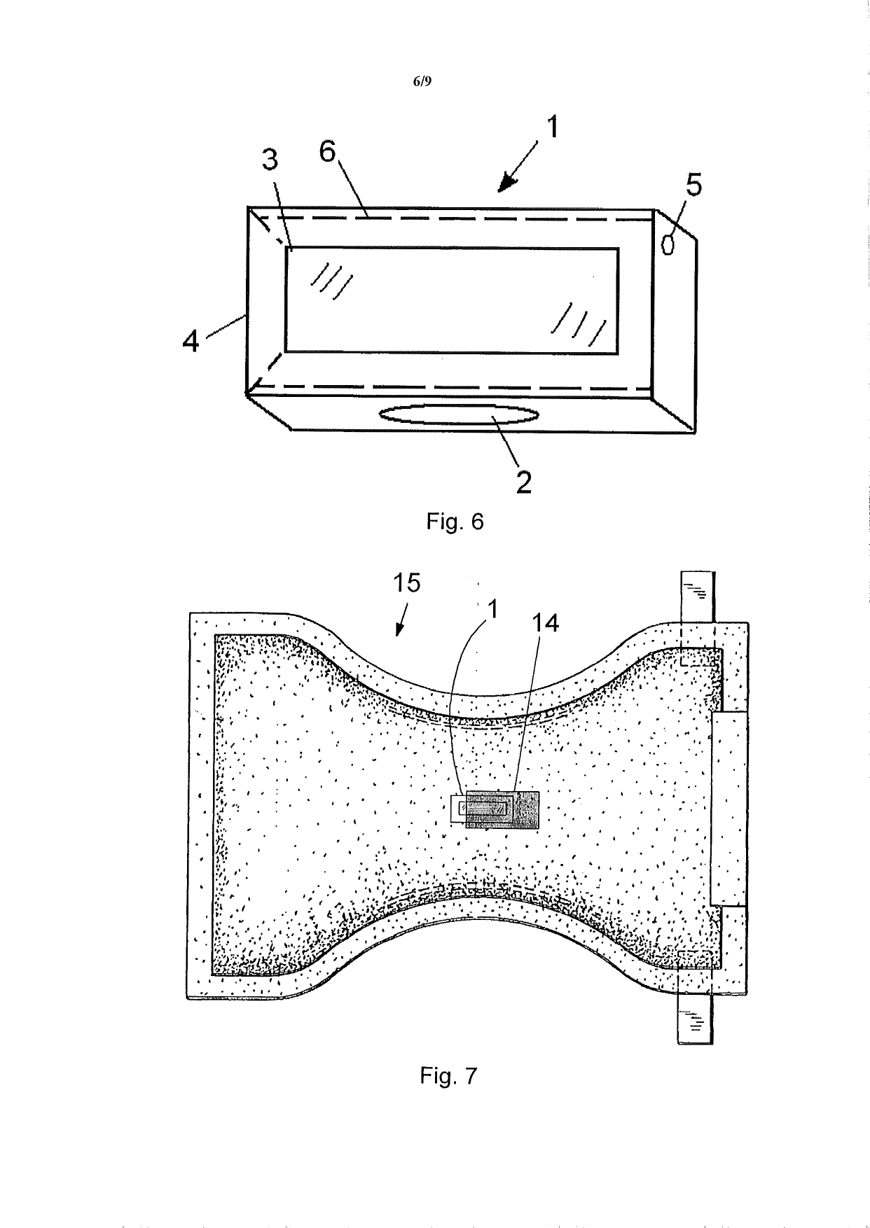

The urine sampling device 1 could also be fitted with fractural impressions 6 as disclosed in FIG. 6. These Impressions would make it easier to dismount the urine sampling device when the gel is to be taken out.

As mentioned above, inside the chamber 3 resides a dry gel that swells when subjected to urine. The gel preferably has inherent properties which are able to preserve a group of bacteria that has followed the urine into the urine sampling device 1, thus preventing substantive growth of the bacteria. This effect has been proven in examples 1 and 2 provided below. The gel can also be mixed with a suitable preservative such as Boric acid, or any other suitable preservative. The preservative provides an even more stable system for preventing growth of the sampled bacteria. When applying a preservative together with a gel having preservative properties of its own, a surprising synergistic effect has been found that provides for an even more effective preservation effect. The gel can preferably be provided in dry form, for example as a powder or a film. A gel made of polyethyleneoxide (PEO) has the mentioned inherent preservative effect. The dry gel is preferably a hydrophilic physically cross linked gel. This gel can consist of polysaccharides such as a CMC based gel. This is considered an advantage as the gel would become biodegradable. The dry gel can also consist of synthetic polymers, preferably hydrophilic polymers. This is advantageous as the polymers can be provided homogenously in composition from batch to batch.

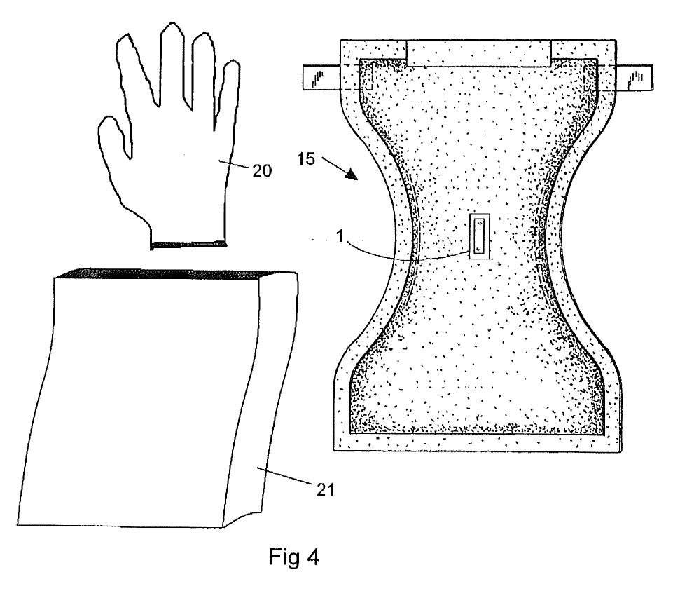

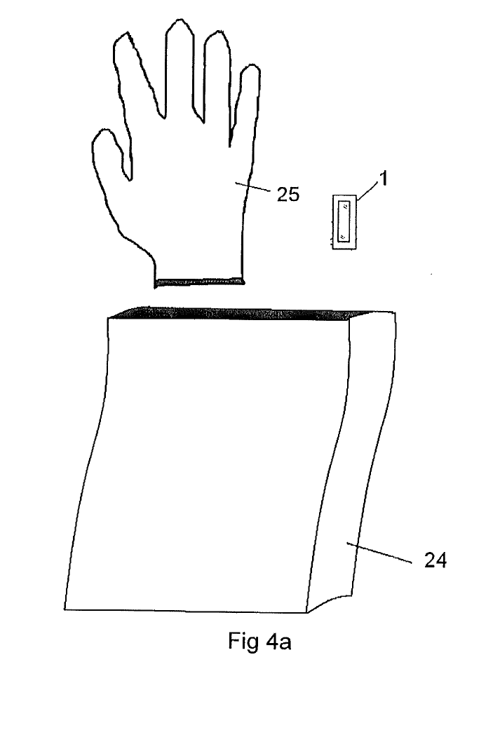

By providing the device in a urine sampling kit as can be seen in FIG. 4a the urine sampling can be simplified. The device is provided with a container 24 for transport to an analysing site. There can also be provided a glove 25. And there can also be provided an absorbent product 15 provided with the urine sampling device 1 preinstalled, as seen in FIG. 4.

### EXAMPLE 1

### Testing the Precision of the System

It is important that the amount bacteria can be correctly determined. The purpose of this test was to ensure that the determined number of bacteria using the urine sampling device does not differ more than acceptable from what is determined directly from a solution

In this test an overnight culture of an E. coli (CCUG 49263, NCTC 10538) was diluted in tenfold steps in Tryptic Soy Broth (TSB) and saline (1:9). Three of these dilutions were measured directly with traditional pour plate technique from the solution (TSA) and compared to the determined amounts when using the urine sampling device.

The urine sampling device was produced as follows: A 5% (w/w) water solution of polyethyleneoxide (PEO) from Scientific Polymer Products (Mw=200 000 g/mole) was produced by dispersing the powder in water and subsequently stirring the solution for at least 12 hrs. The solution was then transferred to a small aluminium mould (diameter 2.5 cm and depth 1.5 cm) and frozen at −80° C. for at least 12 hrs. The aluminum mould was dismounted from the frozen solution before it was put in the freeze dryer (FD 3 from Heto Lab Equipment) until all water was sublimated. The freeze dried PEO was then cut in halves and one half was used in all experiments. When boric acid was used as a preservative it was added to the PEO solution before the freeze drying step.

0.2 ml of E. coli suspension was added to the dry PEO-gel. After some minutes the gel was put in 10 ml NaCl and dissolved (about 10 min). After that the PEO gel was totally dissolved, the amounts of bacteria were determined again with pour plate on TSA.

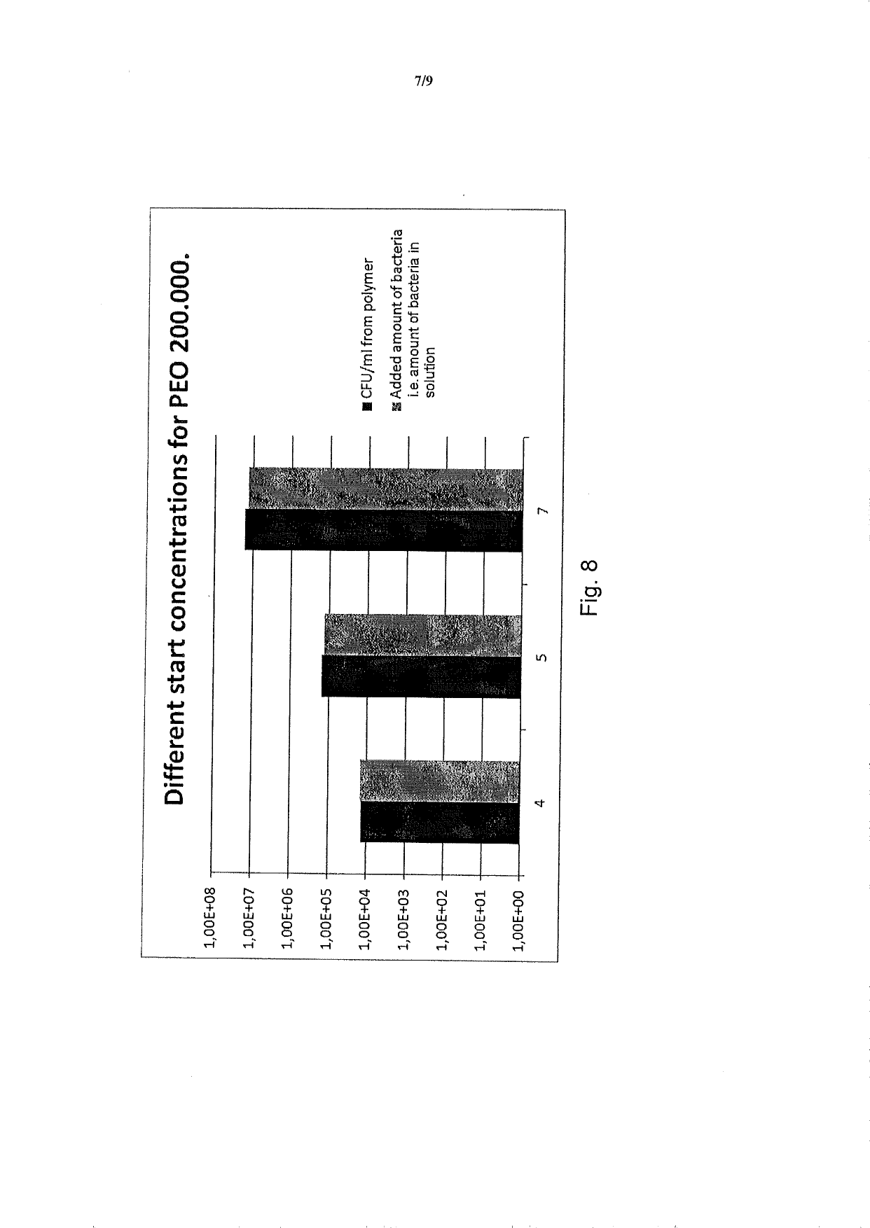

In FIG. 8 the numbers determined directly from suspension are compared to the numbers determined when the suspension was trapped by the dry gel, which was then dissolved in saline and counted afterwards.

As can be seen in the FIG. 8 almost the equal amount of bacteria is determined directly from suspension compared to suspension trapped in PEO gel.

### EXAMPLE 2

### Testing the Stability of the System

The purpose of this test was to ensure that the system is stable and that time between urination and sampling can be at least 12 hours without changing the analyzed result more than +/−one log unit. The gel should preserve the trapped microorganisms without killing them in any significant amount. This can be achieved either in that the polymer can have a sufficient preservative effect in itself or this effect can be further enhanced by using an extra additive with preservative effect.

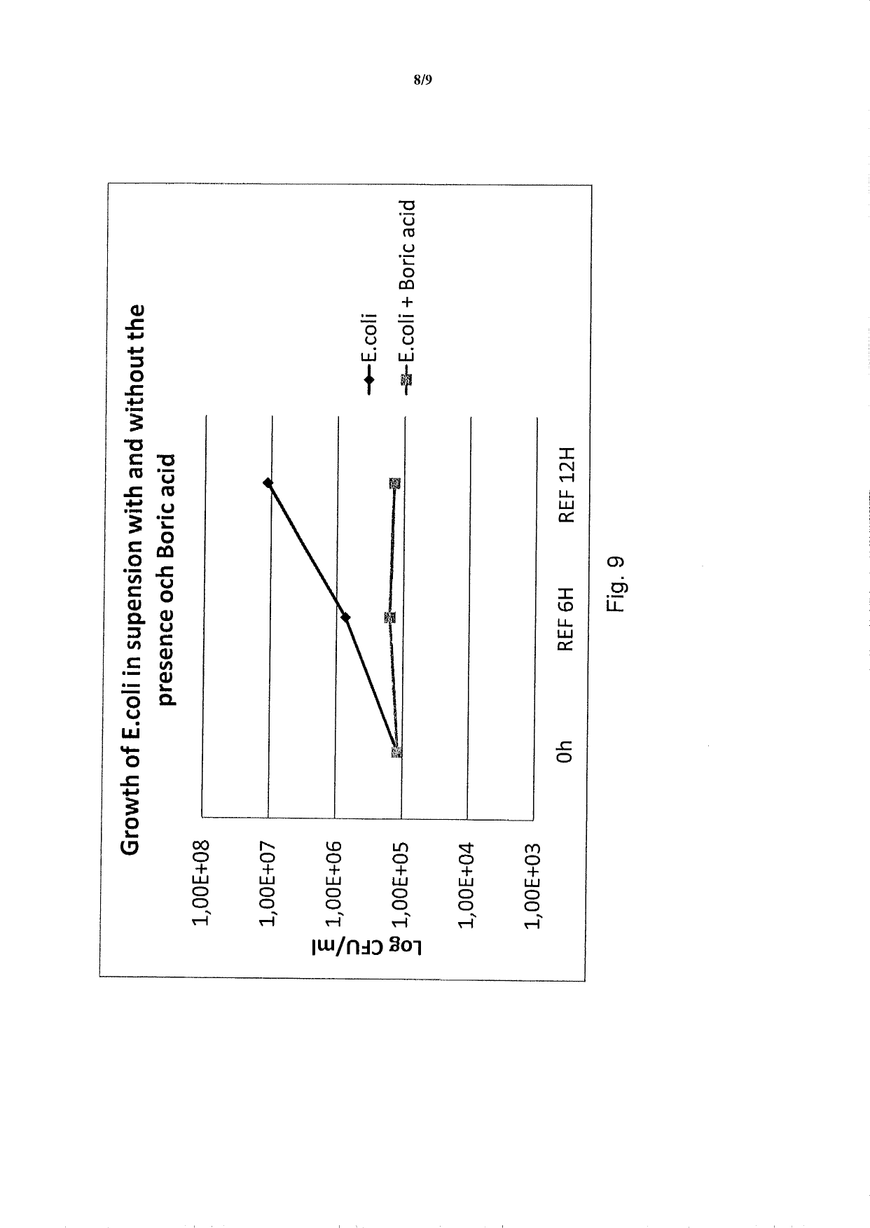

In a first study a suspension with TSB saline (1:9) was inoculated with 10⁵ CFU/ml of an uropathogenic E. coli and allowed to grow for 12 hours under studying. In the FIG. 9 it can be seen that in the E. coli when in suspension grows 2 logs in the non-preserved system and in the system with preservative a stable system is achieved by the addition of 1.8% (w/w) of Boric acid as preservative.

In the second experiment in of example 2, a dried gel with PEO (100 000 g/mole) was poured with 200 μl of TSB saline (1:9) with an E. coli (10⁵ CFU/ml). The gel was taken out directly (after 5 minutes), after 6 hours and after 12 hours. The gel was then dissolved in 10 ml NaCl and analyzed with pour-plate cultivation on TSA.

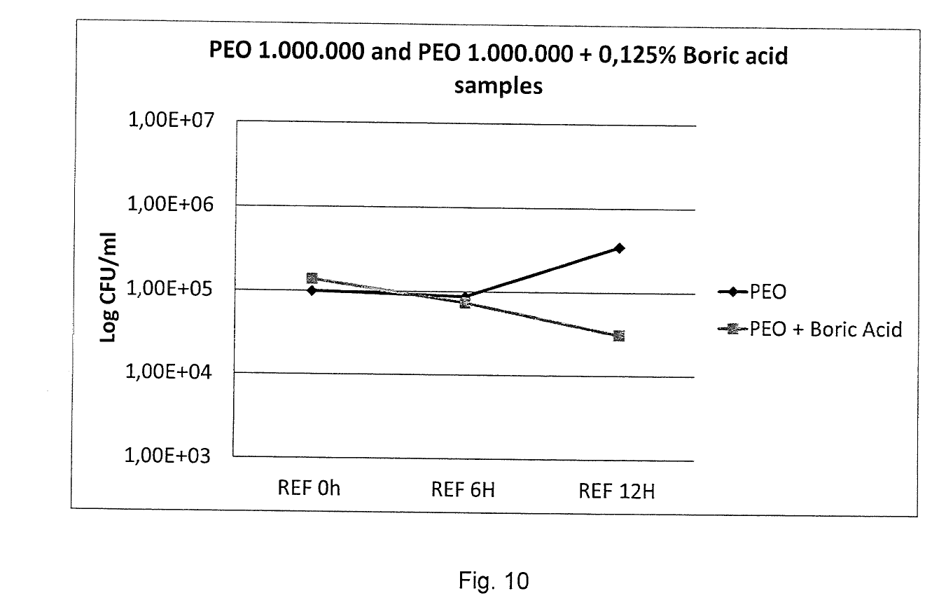

As can be seen in the FIG. 10 the PEO in itself has a preservative effect. The measured increase of E. coli measured after 12 hours is 0.5 log units (compared to two log increase in FIG. 9). With an addition of Boric acid (0.125% (w/w)*) to the dried PEO gel a decrease of 0.5 log units was measured after 12 hours. A deviation of 0.5 log units after 12 hours may be acceptable for this measuring of the urine sampling device. And there is absolutely a potential for further adjustments to make a system that is very stable for at least 12 hours. *The amount of boric acid is denoted as the amount added to the PEO solution and is a rough estimation of the amount necessary in the dry gel, such that the 200 μl of TSB saline (1:9) with E. coli that hits the urine sampling device will encounter 1.8% (w/w) of boric acid.

1. A urine sampling device comprising:
a housing comprising a urine sampling chamber,
the housing of the urine sampling device configured such that the urine sampling device can be associated with a hygiene absorbent product, and the urine sampling device comprises an inlet for fluid,
said urine sampling chamber comprises a dry gel that can be swelled by urine, and wherein said urine sampling device is configured to preserve a bacteria content from a urine sampled in the urine sampling device, either by means of the inherent properties of the dry gel and/or by a preservative in the dry gel.

2. The urine sampling device according to claim 1, wherein said gel is a physically cross linked hydrophilic gel.

3. The urine sampling device according to claim 1, wherein said dry gel is chosen from a group consisting of polysaccharides and synthetic polymers.

4. The urine sampling device according to claim 3, wherein said dry gel is polyethyleneoxide (PEO).

5. The urine sampling device according to claim 1, wherein the dry gel comprises the preservative, and wherein the preservative is a composition that has acidic and/or desiccant properties.

6. The urine sampling device according to claim 1, wherein the dry gel comprises the preservative, and wherein said preservative is chosen from a group consisting of acids and salts.

7. The urine sampling device according to claim 1, wherein the dry gel comprises the preservative, and wherein said preservative is present in an amount such that any bacteria present in a urine sample that has entered said chamber can be held in said chamber for a period of at least 12 hours, where growth of bacteria is less than one log unit and/or death of bacteria is less than one log unit.

8. The urine sampling device according to claim 1, wherein an outer surface of the urine sampling device comprises an attachment means, for attaching the urine sampling device to the body side of an absorbent product.

9. The urine sampling device according to claim 8, wherein said attachment means is configured to detachably attach the urine sampling device to said absorbent product.

10. The urine sampling device according to claim 8, wherein said attachment means is an adhesive, or a hook and loop arrangement.

11. The urine sampling device according to claim 1, wherein said chamber comprises a selected amount of dry gel configured to swell and hold a predetermined volume of urine in the chamber.

12. The urine sampling device according to claim 11, wherein said predetermined volume is 100 μl to 5000 μl.

13. The urine sampling device according to claim 1, further comprising a closing device for closing said inlet to seal the chamber when said urine sampling device has received a predetermined volume of urine.

14. The urine sampling device according to claim 1, wherein the urine sampling device is dismountable by means of fractural impressions made in the housing, such that a urine sample absorbed by said dry gel, can be removed from said chamber, for further examination.

15. The urine sampling device according to claim 1, wherein the housing of the urine sampling device comprises a ventilation opening for ventilating air out of the urine sampling device when the urine sampling device is filled with urine.

16. A hygiene absorbent product, wherein the hygiene absorbent product comprises an associated urine sampling device according to claim 1.

17. The hygiene absorbent product according to claim 16, wherein the hygiene absorbent product comprises means for incorporating the urine sampling device.

18. A urine examination kit, comprising a urine sampling device according to claim 1 and a sample collection container configured to house the urine sampling device, for transport of said urine sampling device for further examination.

19. The urine examination kit of claim 18, further comprising a glove for preventing contamination of the urine sampling device when removing the urine sampling device from the hygiene absorbent product.
